(19) United States
(12) Patent Application Publication
(10) Pub. No.: US 2021/0145492 A1
(43) Pub. Date: May 20, 2021

(54) MEDICAL PLATE AND LOCKING FASTENER WITH DIFFERENT THREAD PITCHES

(71) Applicant: Orchid MPS Holdings, LLC, Holt, MI (US)

(72) Inventor: Ernest N. Corrao, JR., Bethel, CT (US)

(21) Appl. No.: 16/949,724

(22) Filed: Nov. 12, 2020

Related U.S. Application Data
(60) Provisional application No. 62/950,154, filed on Dec. 19, 2019.
(60) Provisional application No. 62/937,925, filed on Nov. 20, 2019.

(51) Int. Cl. A61B 17/80; A61B 17/86

(52) U.S. Cl. CPC A61B 17/8057; A61B 17/86

(57) ABSTRACT

A surgical implant assembly includes a plate having at least one aperture extending through the plate and having a central axis. A screw includes a head and a body, with the head including at least one head thread configured to engage with the plate at the aperture and the body including at least one body thread configured to engage a bone material. The at least one head thread has a first pitch and the at least one body thread has a second pitch that is different than the first pitch. When the plate is disposed at a surface of the bone material and the screw is rotatably driven into the bone material, the pitch difference enables the plate to one selected from the group consisting of (i) lift away from the surface of the bone material and (ii) compress down onto the surface of the bone material.

### CROSS REFERENCE TO RELATED APPLICATIONS

The present application claims the filing benefits of U.S. provisional application Ser. No. 62/950,154, filed Dec. 19, 2019, and U.S. provisional application Ser. No. 62/937,925, filed Nov. 20, 2019, which are hereby incorporated herein by reference in their entireties.

### FIELD OF THE INVENTION

The present disclosure relates generally to medical plates and fasteners.

### BACKGROUND OF THE INVENTION

In order to help mend fractured or broken bones or to assist in correcting deformities, a physician, or other suitable healthcare provider, may position the fractured or broken bone into its normal alignment and hold the bone together with a surgical implant. In some implementations, these surgical implants include a plate and fasteners, such as, for example, screws, nails, wires, etc., that extend through or from the plate and secure the plate to the bone. Often, these surgical implants are internally fixated to the fractured or broken bone, which may result in surgical procedures occurring in tight spaces and at difficult angles.

### SUMMARY OF THE INVENTION

The surgical implant assembly described herein provides a variable angle locking fastener and plate for enhanced securing and mending of fractured bones or for correcting deformities. The surgical implant assembly includes a plate and a first screw. The plate includes at least one aperture extending through the plate between opposite surfaces of the plate. The screw comprises a head and a body, the head including at least one head thread configured to engage with the plate at the aperture and the body including at least one body thread configured to engage a bone material.

According to an aspect of the present invention, the at least one head thread has a first pitch and the at least one body thread has a second pitch that is different than the first pitch. When the plate is disposed at a surface of the bone material and the screw is rotatably driven into the bone material, the pitch difference between the first pitch of the at least one head thread of the head and the second pitch of the at least one body thread of the body enables the plate to one selected from the group consisting of (i) lift away from the surface of the bone material and (ii) compress down onto the surface of the bone material. Optionally, for example, a plate may be provided and if it is desired to have the plate spaced from the bone material, a first fastener or screw may be selected that lifts the plate away from the surface of the bone material as the first screw is rotatably driven into the bone material. And if it is desired to have the plate tight against the bone material, a second fastener or screw may be selected that compresses the plate down onto the surface of the bone material as the second screw is rotatably driven into the bone material. Optionally, a third fastener or screw may be provided and selected with the head pitch substantially matching the body pitch, such that the plane neither lifts away from the bone material or compresses onto the bone material as the third screw is rotatably driven into the bone material.

According to another aspect of the present invention, the head of the screw includes a plurality of head threads configured to engage with the plate at the aperture. The plurality of head threads provide a plurality of thread entry points for threading into the plate. The plurality of head threads are configured to, when the plate is disposed at a surface of the bone material and the screw is rotatably driven into the bone material, enable the plate to one selected from the group consisting of (i) lift away from the surface of the bone material, (ii) compress down onto the surface of the bone material, and (iii) neither compress down onto the surface of the bone material nor lift away from the surface of the bone material.

According to another aspect of the present invention, the plate includes a plurality of circumferentially spaced apart protrusions established at and extending into the aperture. Each of the plurality of protrusions includes a plurality of buttresses. The buttresses of each protrusion include at least two buttresses spaced apart between the opposite surfaces of the plate, with each of the buttresses being parallel to the opposite surfaces of the plate. The screw includes a head and a body. The head includes at least one head thread configured to engage with the plurality of buttresses.

The buttresses of the plate are configured to allow for the screw to be rotatably driven into the plate (and into the bone of a patient) at different angles, without cross-threading of the threads of the screw in the plate. The spaced apart stepped buttresses provide a series of steps or parapets or castellations spaced so that the thread of the screw can pass through the spaces between adjacent ones of the steps or parapets or castellations at various angles, and thus allow for threads of the screw head to be received at different spaces between the buttresses or parapets to allow for the screw to be effectively rotatably driven into the plate at different angles. The thread pitch of the plate-engaging head threads of the screw is selected to provide lifting of the plate away from the bone as the screws are fully driven into and seated at the plate or compression of the plate at the bone as the screws are fully driven into and seated at the plate, or a neutral securing of the plate at the bone.

Optionally, the spaced apart buttresses may be formed at the screw head, and the threads (or spaced apart partial threads) may be formed in the plate, such that the engagement of the buttresses of the screw with the threads of the plate allow for the screw to be rotatably driven through the plate at different angles.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

FIG. 1 is a perspective view of an implant assembly that includes a plate and screws in accordance with the principles of the present disclosure;

FIG. 2 is a side elevation view of the implant assembly of FIG. 1 with the screws secured to a medium, such as a bone;

FIG. 3 is a perspective view of the plate of the implant assembly of FIG. 1;

FIG. 4 is a top plan view of the plate of the implant assembly of FIG. 1;

FIG. 5A is a cross-sectional view of the plate of FIG. 4, taken along line A-A;

FIG. 5B is a cross-sectional view of the plate of FIG. 4, taken along line B-B;

FIG. 5C is a cross-sectional view of the plate of FIG. 4, taken along line C-C;

FIG. 5D is an enlarged portion of the cross section of FIG. 5A;

FIG. 6 is a top plan view of the plate of the implant assembly of FIG. 1, showing exemplary dimensions for the plate;

FIG. 7A is a side cross-sectional view of the plate of FIG. 6, taken along line A-A, showing exemplary dimensions for the plate;

FIG. 7B is a side cross-sectional view of the plate of FIG. 6, taken along line B-B, showing exemplary dimensions for the plate;

FIG. 7C is a side cross-sectional view of the plate of FIG. 6, taken along line C-C, showing exemplary dimensions for the plate;

FIG. 8A is a perspective view of one of the screws of the implant assembly of FIG. 1;

FIG. 8B is a top plan view of one of the screws of the implant assembly of FIG. 1;

FIG. 9 is a side elevation view of one of the screws of the implant assembly of FIG. 1;

FIG. 10 is a side cross-sectional view of the screw of FIG. 9, taken along line A-A;

FIG. 11A is another side cross-sectional view of the screw of FIG. 9, taken along line A-A, showing exemplary dimensions for the screw;

FIG. 11B is an enlarged view of the portion B of the screw of FIG. 11A;

FIG. 12 is a side partial-cross-sectional view of the plate and one of the screws of the implant assembly of FIG. 1, shown with the screw aligned with a central axis of the aperture of the plate;

FIG. 13 is a side partial-cross-sectional view of the plate and one of the screws of the implant assembly of FIG. 1, shown with the screw angled or tilted relative to the central axis of the aperture of the plate;

FIG. 14 is a perspective view of another implant assembly that includes a plate and screws in accordance with the principles of the present disclosure;

FIG. 15 is a perspective view of the plate of the implant assembly of FIG. 14;

FIG. 16 is a side elevation view of one of the screws of the implant assembly of FIG. 14;

FIG. 17 is a perspective view of one of the screws of the implant assembly of FIG. 14;

FIG. 18 is a top plan view of the plate of the implant assembly of FIG. 14; and

FIG. 19 is a top plan view of one of the screws of the implant assembly of FIG. 14.

### DETAILED DESCRIPTION

Example configurations will now be described more fully with reference to the accompanying drawings. Example configurations are provided so that this disclosure will be thorough, and will fully convey the scope of the disclosure to those of ordinary skill in the art. Specific details are set forth such as examples of specific components, devices, and methods, to provide a thorough understanding of configurations of the present disclosure. It will be apparent to those of ordinary skill in the art that specific details need not be employed, that example configurations may be embodied in many different forms, and that the specific details and the example configurations should not be construed to limit the scope of the disclosure.

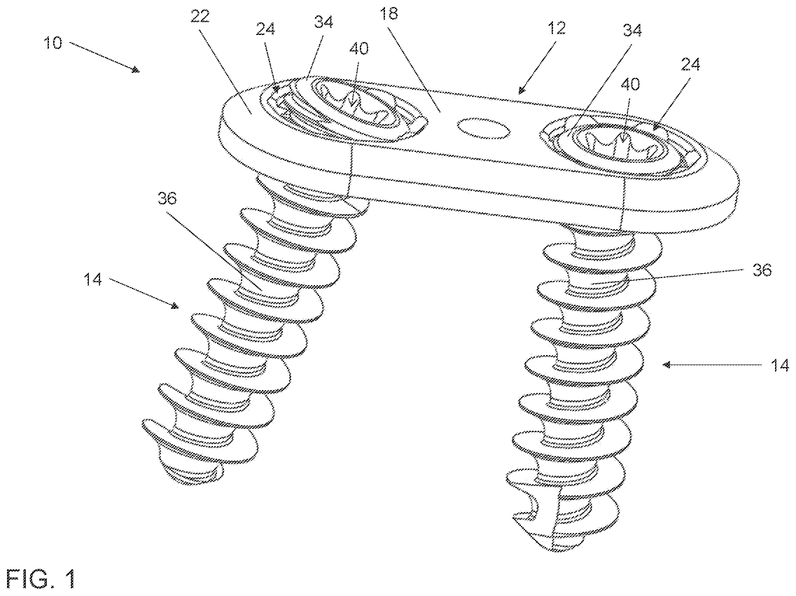

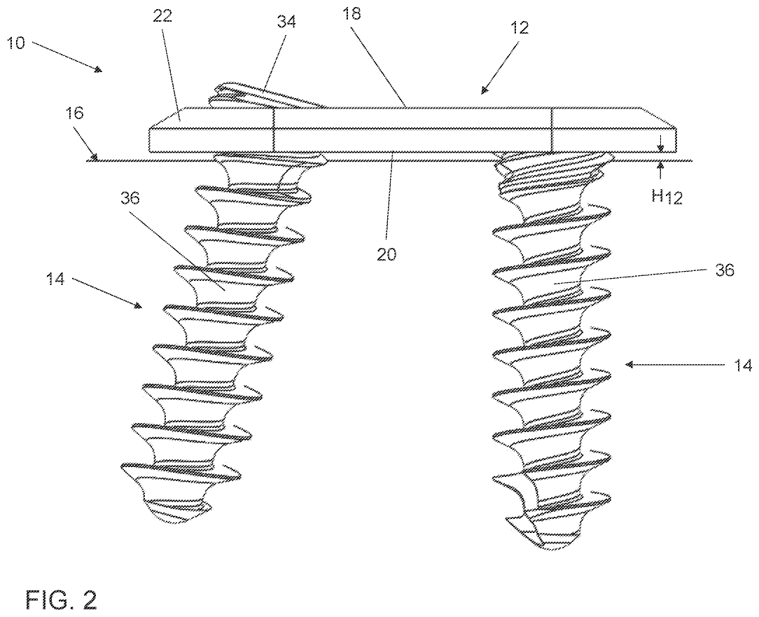

Referring to FIGS. 1 and 2, a surgical implant assembly 10 generally includes a plate 12 and a pair of fasteners or screws 14. The implant assembly 10 may be used during a surgical procedure to, for example, maintain the positioning of broken or fractured bone pieces such that the broken or fractured bone may heal in the proper positioning and alignment. Such a surgical procedure may be conducted by a physician or any other suitable healthcare provider. The implant assembly 10 is implanted in a patient's body with the screws 14 being screwed into bone material 16 of a patient and the plate 12 being either spaced from the bone material 16 (as shown in FIG. 2) or abutting the bone material 16. The threads of the screws and the configuration of the plate cooperate to allow the screws to be rotatably driven into the plate and bone at different angles and to provide a desired degree of compression of the plate at the bone or a desired gap between the plate and the bone, as discussed below.

For example, the plate 12, with the screws screwed into the bone and the head threads engaging the plate 12 (as also discussed below), may be spaced from the bone material 16 by a height or gap H₁₂ which may be less than 0.5 mm, such as, for example, approximately 0.3 mm (such as shown in FIG. 2). The plate 12 being spaced from the bone material 16 may help to prevent necrosis under the plate 12 and/or damage to periosteal tissue. Due to the surgical procedure being performed internally, situations may arise where screwing the screws 14 into the bone material 16 must be accomplished in tight spaces or at less-than-ideal angles.

Referring to FIGS. 1-7C, 12, and 13, the plate 12 includes a top surface 18 and a bottom surface 20 spaced from the top surface 18. The top surface 18 and the bottom surface 20 may be generally flat or planar and generally parallel to one another (or may be curved or complex curvatures or shapes to adapt or conform or correspond with the patient anatomy). In some implementations, the top surface 18 includes a chamfer 22 extending around a periphery of the top surface 18. The chamfer 22 may extend at an angle of between 15 degrees and 60 degrees relative to the top surface 18, such as, for example, an angle of about 30 degrees relative to the top surface 18, or the periphery of the top surface may be rounded and lacking a sharp edge.

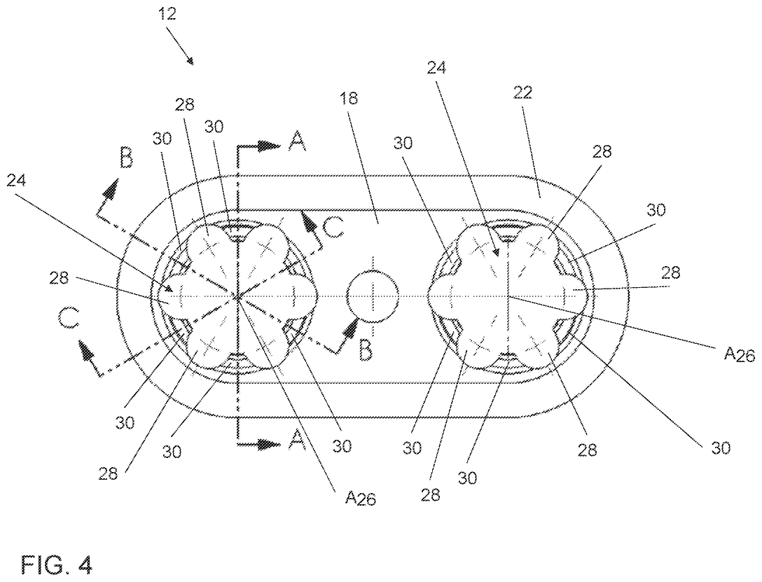

The plate 12 has a pair of apertures 24 extending therethrough. While two apertures 24 are shown and described, it should be understood that any suitable number of apertures may be implemented. For example, the plate 12 may include a single aperture 24 or three or more apertures (and the plate may be used with a corresponding number of screws or fasteners 14), depending on the particular application. As shown in FIG. 4, each of the apertures 24 comprises a central aperture 26 and a plurality of radial apertures 28 circumferentially or peripherally spaced around the periphery of the central aperture 26, with a plurality of protrusions 30 formed between the radial apertures 28. The plate 12 may have six radial apertures 28 and six protrusions 30 (such as shown), or may have any suitable number of radial apertures 28 and protrusions 30.

The central aperture 26 may include a central axis A₂₆ and the radial apertures 28 may be formed with central axes parallel to the central axis A₂₆, as can be seen in FIG. 4. In some implementations, the radial apertures 28 may be formed at any suitable angle relative to the central axis A₂₆, such as, for example, about 14 degrees relative to the central axis A₂₆ to correspond to the angle at which the protrusions and buttresses are formed, as discussed below.

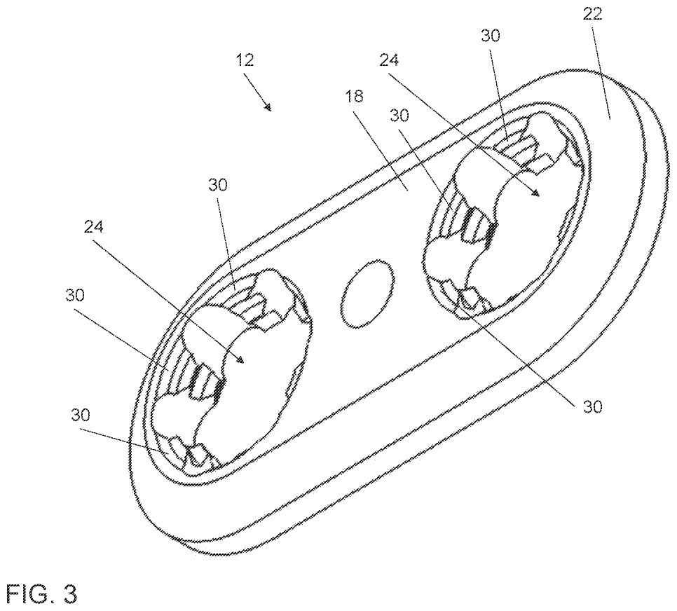

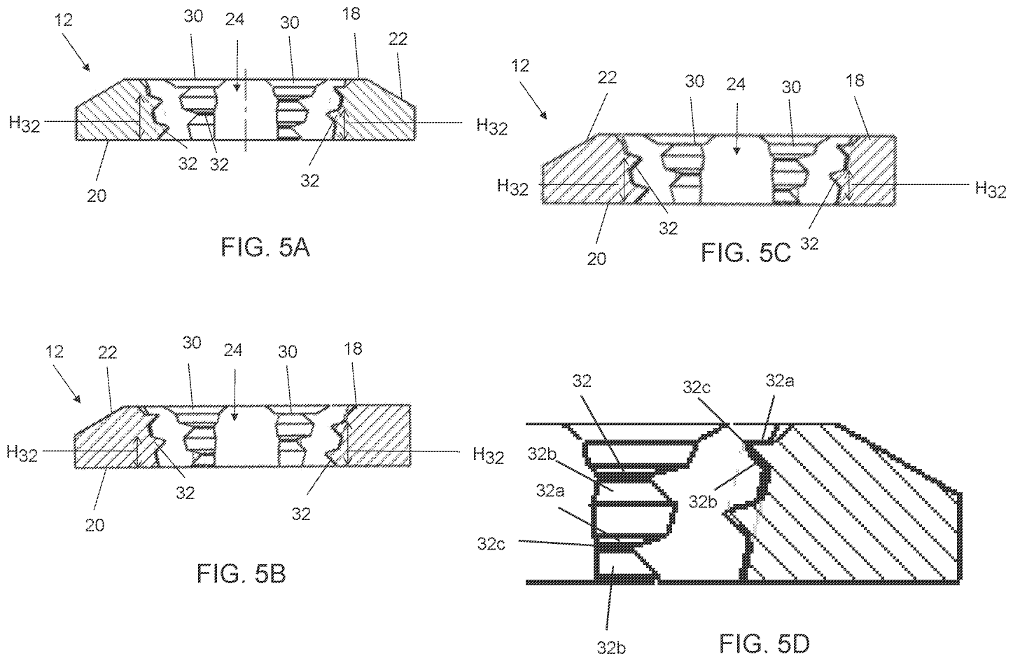

The plurality of protrusions 30 are formed between the radial apertures 28 so that they extend or protrude into and partially define the shape of the larger aperture 24 but do not extend or protrude into the central aperture 26. As shown in FIGS. 3-7C, 12, and 13, each of the protrusions 30 includes a plurality of buttresses or steps or parapets or castellations 32 that are vertically spaced apart between the top and bottom of the plate 12. The buttresses 32 are parallel to one another and are generally parallel to the top surface 18 and the bottom surface 20 of the plate 12 at the aperture and are generally normal to or perpendicular to the central axis of the aperture. In other words, and as can be seen with reference to FIG. 5D, each buttress 32 comprises a tapered tooth or ridge that has an upper surface 32a and a lower surface 32b that meet at an edge 32c, with the edges of the buttresses being parallel to one another and to the top and bottom surfaces or planes of the plate (i.e., an end of a buttress edge is at the same height above the lower surface of the plate as the opposite end of that buttress edge). The buttresses of each protrusion are vertically spaced apart from one another and are circumferentially spaced from the buttresses of the other protrusions and allow the screw to rotatably engage the plate (as discussed below). That is, the edges of the buttresses are perpendicular to the central axis of the aperture and are not angled relative to the top or bottom surface of the plate and thus do not extend in a swirling or helical configuration found in typical threads. For curved plates or complex curved plates, the central axis of the aperture extends normal to or perpendicular to the plate surface at that location, with the buttresses formed along the aperture and normal to or perpendicular to the central axis of the aperture.

Each of the buttresses 32 of each protrusion 30 is formed at a respective height H₃₂ relative to the bottom surface 20 of the plate 12 (FIGS. 5A-C), with the height H₃₂ of one of the buttresses 32 of a particular protrusion being different than the height H₃₂ of another one of the buttresses 32 of the same protrusion 30. Also, the height H₃₂ of one of the buttresses 32 (e.g., an upper one of the buttresses) of one protrusion is different than a height H₃₂ of a corresponding one of the buttresses 32 (e.g., the upper one of the buttresses) on a different or adjacent protrusion 30. For example, the height H₃₂ of the upper one of the buttresses 32 of one protrusion 30 is different than a height H₃₂ of the upper one of the buttresses 32 of an adjacent protrusion 30, such that the positioning of the buttresses 32 may provide a stepped configuration around the aperture without the buttresses 32 themselves being angled in a helical manner.

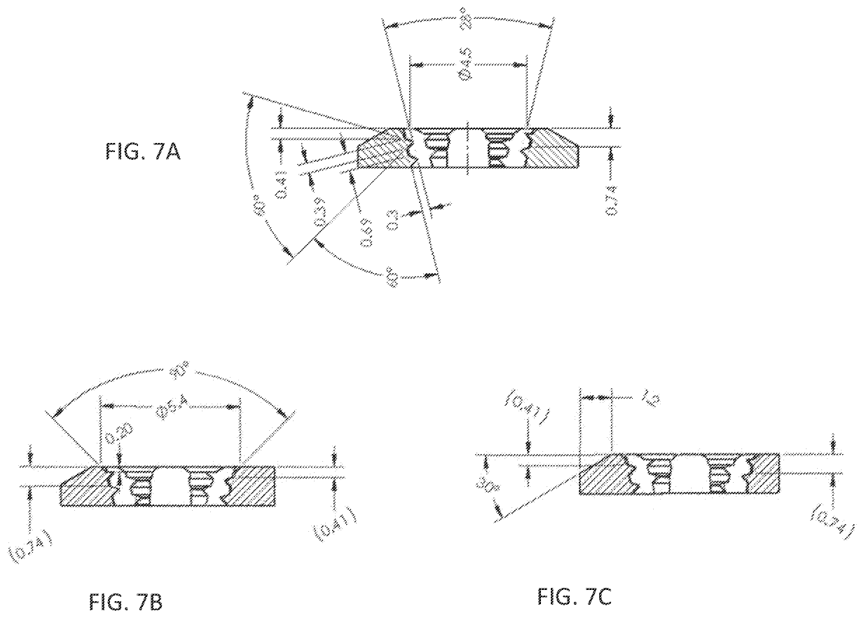

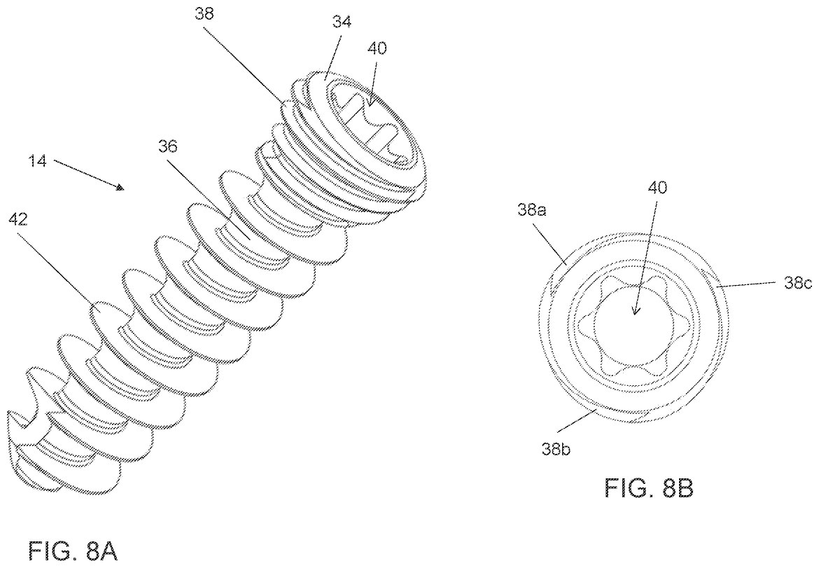

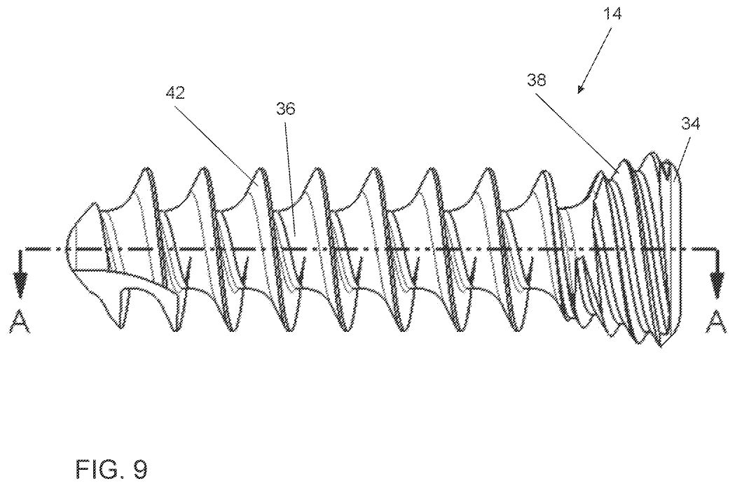

The central aperture 26 has a central axis A₂₆ and the buttresses 32 of each protrusion 30 may be formed at an angle relative to the central axis A₂₆. For example, and such as shown in FIG. 7A, the buttresses 32 may be formed or established at the protrusions along an angle of approximately 14 degrees relative to the central axis A₂₆, such that the upper buttresses of diametrically opposite protrusions are spaced diametrically further apart than the lower buttresses of the diametrically opposite protrusions. The buttresses may be formed at any suitable angle relative to the central axis A₂₆.

The buttresses thus cooperate to form a plurality of parallel steps or parapets or castellations (that are parallel to one another and parallel to the top and/or bottom surface of the plate or normal to the central axis A₂₆ of the aperture of the plate) that function to provide a pseudo-helical configuration for the threads of the screw to engage. In other words, the steps or buttresses provide a series of parapets or castellations that are spaced apart so that a thread of the screw can pass through the notches between adjacent ones at various angles. The buttresses 32 thus cooperate with one another to effectively provide multiple potential thread paths for the threads of the fastener or screw 14 so that the screw 14 may be rotatably driven into the plate 12 at different angles, as discussed below.

The apertures 24 and buttresses 32 may be formed by any suitable process and in any suitable manner. For example, the plate 12 may be formed as a solid, unitary component and then the apertures 24 may be subsequently formed in the plate 12. For example, the central aperture 26 may be formed or drilled through the plate and then the radial apertures may be formed or drilled through the plate around the central aperture, leaving the protrusions between the radial apertures and outboard of the central aperture, whereby each of the protrusions may be machined or ground to form the respective vertically spaced apart buttresses. As another example, the apertures 24 and protrusions and buttresses 30 may be formed approximately simultaneously with the plate 12 via, e.g., forging, stamping, molding, or the like.

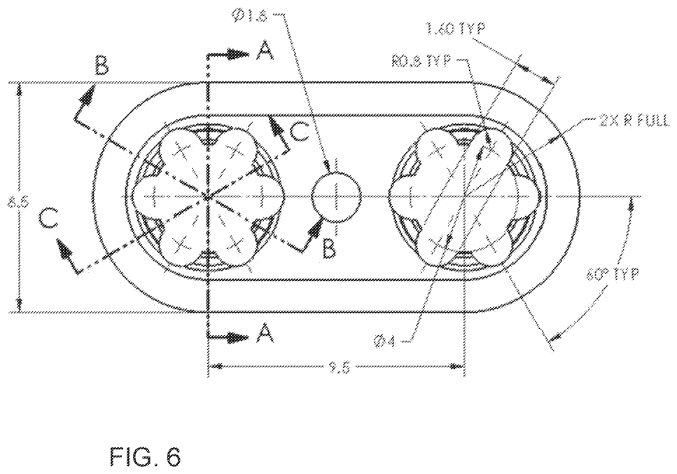

FIGS. 6-7C illustrate exemplary dimensions of the plate 12 and should not be construed as the only possible configuration of the plate 12. Further, it should be understood that the dimensions shown in these figures may be modified as necessary and include any suitable tolerances. FIGS. 6-7C do not include reference numerals for improved clarity, however, it should be understood that the features illustrated in these figures are the same as those shown in FIGS. 4-5C.

In the illustrated embodiment, the implant assembly 10 includes two screws 14 for rotatably driving into a respective aperture 24 of the plate 12 and into the bone of the patient. Each of the screws 14 includes a head 34 and a threaded body 36 extending from the head 34. The head 34 and the body 36 are generally cylindrical and formed around a central axis A₁₄. The head 34 includes a plurality of head threads or entry threads 38 and the body 36 has one or more body threads 42. The head 34 also includes a drive or socket 40 that is configured to receive or otherwise engage a tool or driver to rotatably drive the screw 14 into the bone and plate. The socket 40 may be any suitable socket to receive any suitable tool, such as, for example, a star driver, an Allen wrench, a screwdriver, or the like. The body thread 42 is sized to be received through the central aperture 26 of the plate and is configured to thread into the bone of the patient while the head threads 38 are configured to engage the buttresses 32 of the aperture 24 of the plate 12, as discussed below.

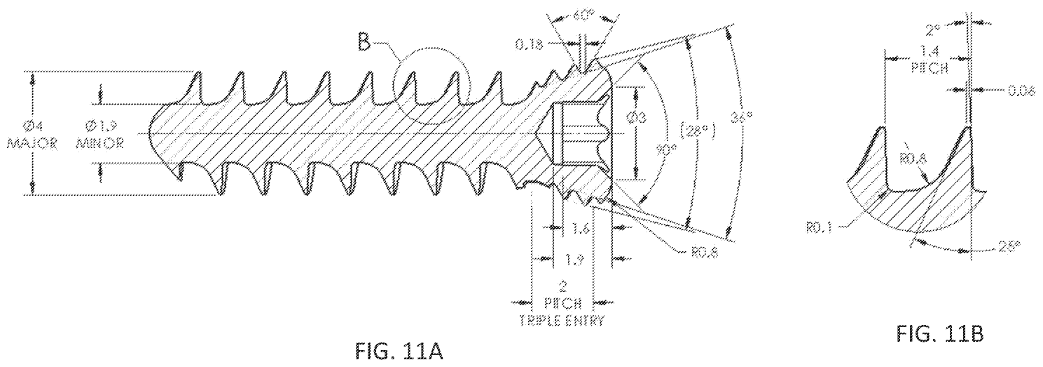

The head threads 38 are formed with an outer edge of the threads 38 being at an angle relative to the central axis A₁₄, such as, for example, an angle of approximately 14 degrees relative to the central axis A₁₄ (so as to generally correspond with the angle of the buttresses formed along the aperture 24), as can be seen in FIG. 11A. The head threads 38 have a pitch P₃₈ that is greater than the pitch of the body thread 42. For example, the head threads 38 may have a pitch P₃₈ that is between 1.5 mm and 2.5 mm, such as, for example, approximately 2 mm, or any other suitable pitch.

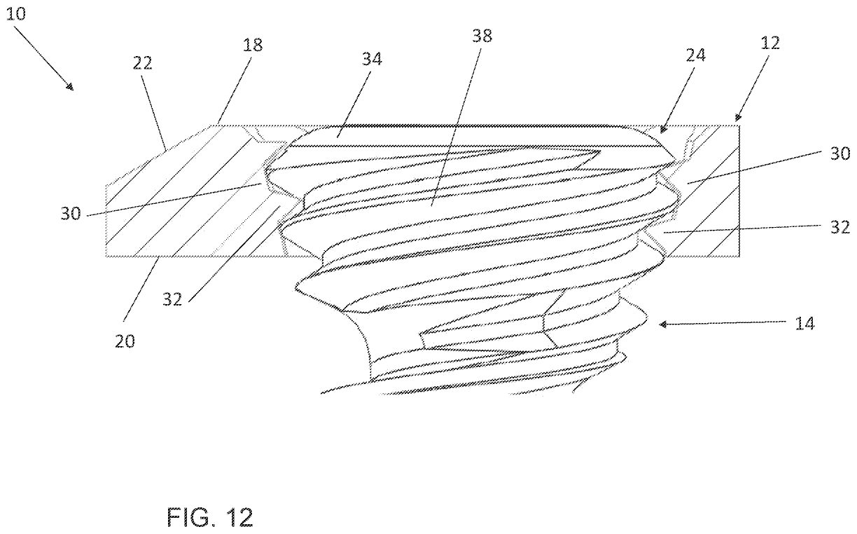

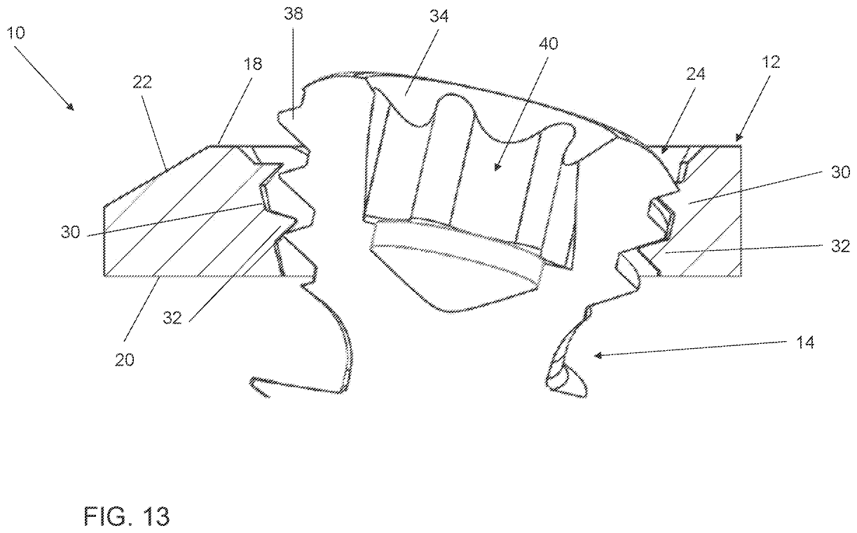

The head threads 38 are configured to engage the buttresses 32 of the protrusions 30 when the screw 14 is inserted through the aperture 24 of the plate 12. The head threads comprise one or more threads, such as two threads or three threads. In the illustrated embodiment, the head threads 38 comprise three head threads or entry threads and include a first entry thread 38a, a second entry thread 38b, and a third entry thread 38c. The entry threads 38a-c allow the screws 14 to engage the buttresses 32 at any one or more of the entry threads 38a-c, which, in conjunction with the varying heights H₃₂ of the buttresses 32, allows the screws 14 to engage the plate straight in (i.e., with the central axis of the screw 14 being parallel to the central axis of the aperture 26 of the plate, such as shown in FIG. 12) or at an angle relative to the plate 12 (i.e., with the central axis A₁₄ of the screw 14 at an angle relative to or not parallel to the central axis A₂₆ of the central aperture 26 of the plate 12, such as shown in FIG. 13). For example, the screw 14 may be rotatably driven into the plate at an angle of between 0 and 15 degrees relative to the central axis A₂₆ of the central aperture 26.

Such a configuration allows a physician or other healthcare provider to screw or thread the screws 14 into the bone material 16 at any suitable angle, such as, for example, an angle between 0 and 15 degrees relative to the central axis of the aperture 26 of the plate, which extends normal to or perpendicular to the plane of the plate. Each screw 14 may operate independently of the other screw 14, i.e., one of the screws 14 may be at an angle of 0 degrees relative to the central axis A₂₆ of the central aperture 26 and the other screw 14 may be at an angle of about 15 degrees (or other angle depending on the configuration of the buttresses of the plate and the head thread pitch) relative to the central axis A₂₆ of the central aperture 26 of the plate 12, as can be seen in FIGS. 1, 2, 12, and 13.

The body 36 of the screw includes one or more body threads 42 (with the illustrated embodiment having a single body thread 42) extending around the central axis A₁₄ of the screw 14 and along the body of the screw. The body thread 42 is formed with a pitch P₄₂ that is, in some implementations, less than the pitch P₃₈ of the head threads 38. The pitch P₄₂ of the body threads 42 may be different than the pitch P₃₈ of the head threads 38 so that the plate 12 may be spaced from the bone material 16 (if the pitch of the body threads is less than the pitch of the head threads) or may be drawn into tighter contact with the bone (if the pitch of the body threads is greater than the pitch of the head threads) when the screws 14 are screwed into the bone material 16. By having the pitch of the body threads be less than the pitch of the head threads, the screws may operate opposite to a compression screw in that the plate 12 may lift slightly from the bone as the body threads are screwed into the bone and the head threads 38 engage the buttresses 32 and lock the screw 14 to the plate 12 when the screw head is fully seated at or fully driven into the plate. Optionally, the pitches of the head threads and the body threads may correspond, whereby the plate compression/lift may be neutral and the plate will be affixed at the bone at the degree of pressure at the bone that was set prior to the head threads engaging the buttresses.

In some implementations, the pitch P₄₂ of the body threads 42 is between 0.9 mm and 1.9 mm, such as, for example, approximately 1.4 mm. The height or gap H₁₂ between the plate 12 and the bone material 16 (when the screws are screwed into the bone and fully seated into the plate) can be calculated by subtracting the pitch P₄₂ of the body threads 42 from the pitch P₃₈ of the head threads 38 and dividing that difference by 2. For example, if the pitch P₃₈ of the head threads 38 is equal to 2 mm and the pitch P₄₂ of the body threads 42 is equal to 1.4 mm, then the gap or height H₁₂ (the gap between the lower surface of the plate and the bone) would be equal to 0.3 mm (2−1.4=0.6, and 0.6/2=0.3 mm).

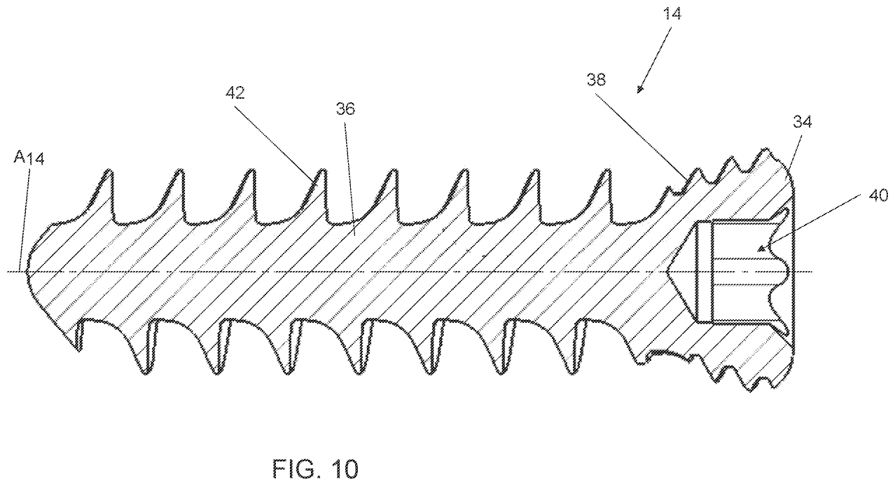

FIGS. 11A and 11B illustrate exemplary dimensions of the screws 14 and should not be construed as the only possible configuration of the screws 14. Further, it should be understood that the dimensions shown in these figures may be modified as necessary and include any suitable tolerances. FIGS. 11A and 11B do not include reference numerals for improved clarity, however, it should be understood that the features illustrated in these figures are the same as those shown in FIG. 10.

During use of the screws and plate, the plate is positioned at a fractured bone and the screws may be threaded into the bone (at opposite sides of the fracture) to affix the plate at the bone. The body thread of each screw is received through the central aperture of the plate and threaded into the bone (optionally via a self-tapping threading process or via threading into a pilot hole drilled into the bone). As the screw is threaded further into the bone, the head threads engage the stepped buttresses of the plate. Due to the different pitches of the body thread and head threads, the head threads function to move the plate relative to the bone as the screw is further screwed into the bone and plate and fully seated or screwed into the plate. For example, with the pitch of the head threads or entry threads being greater than the pitch of the body thread, the plate is drawn back from or raised from the bone as the screw is further screwed into the bone and plate and fully seated or screwed into the plate.

Therefore, the screw and plate configuration allows for the screw or screws to be threaded into the bone of the patient and driven into the plate at different angles. The screw and plate configuration, with the pitch of the head threads being greater than the pitch of the body thread, provides for spacing the plate slightly away from the bone when the screws are fully driven into the plate and into the bone. The screw and plate configuration thus provides for enhanced attachment of the plate to the bone of a patient that allows for different angles of the screws and that may reduce or prevent necrosis under the plate and/or damage to periosteal tissue.

By providing a plate with fasteners having different head thread pitches (relative to the body pitch), the plate and fastener assembly or kit or system can be used in different applications, depending on the desired end result. For example, if it is desired to have a gap between the plate and the surface of the bone, a fastener may be selected that has its head thread pitch greater than the body thread pitch. Alternatively, if it is desired to have the plate tight against the surface of the bone, a fastener may be selected that has its body thread pitch greater than the head thread pitch. The plate and fasteners may be provided as a unit or kit, whereby the healthcare provider can select the appropriate fastener or screw for the particular patient application.

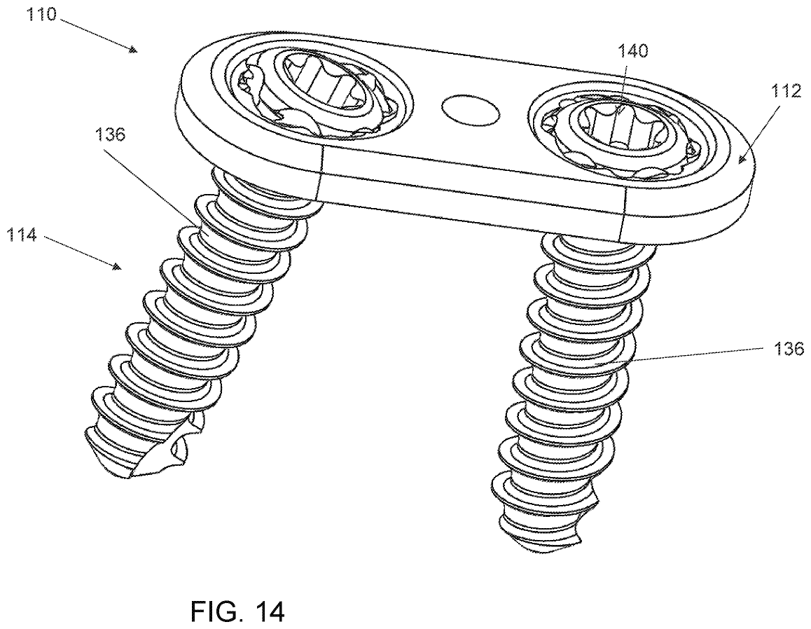

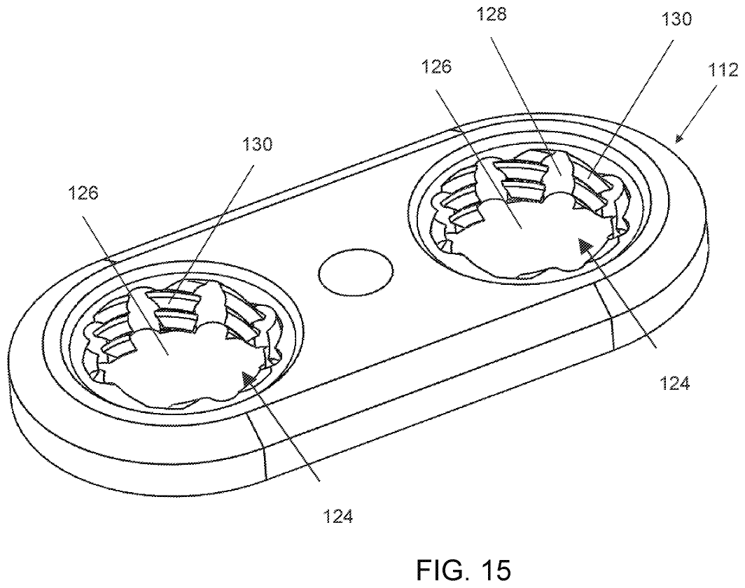

Although shown and described as having spaced apart buttresses or castellations formed in and along the passageway of the plate, with the threads of the screw engaging the buttresses as the screw is rotatably driven into the bone and plate, it is envisioned that the screw head may have spaced apart buttresses or castellations formed around the head, with the plate having threads or partial threads formed in and along the passageway. For example, and such as shown in FIGS. 14-19, a surgical implant assembly 110 generally includes a plate 112 and a pair of fasteners or screws 114. The implant assembly 110 may be used during a surgical procedure to, for example, maintain the positioning of broken or fractured bone pieces such that the broken or fractured bone may heal in the proper positioning and alignment. The implant assembly 110 is implanted in a patient's body with the screws 114 being screwed into bone material of a patient and the plate 112 being either spaced from the bone material or abutting the bone material (such as in a similar manner as discussed above). The configurations of the screws and of the plate cooperate to allow the screws to be rotatably driven into the plate and bone at different angles and to provide a desired degree of compression of the plate at the bone or a desired gap between the plate and the bone, such as discussed above.

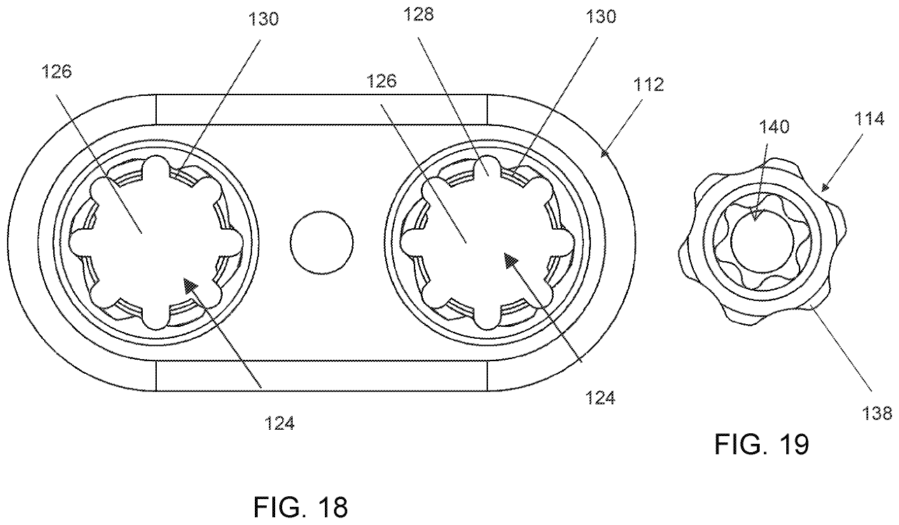

The plate 112 has a pair of apertures 124 extending therethrough. As shown in FIG. 18, each of the apertures 124 comprises a central aperture 126 and a plurality of radial apertures 128 circumferentially or peripherally spaced around the periphery of the central aperture 126, with a plurality of partial threads 130 formed between the radial apertures 128. The plate 112 may have eight radial apertures 128 and eight sets of protrusions or partial threads 130 (such as shown), or may have any suitable number of radial apertures and partial threads. The partial threads cooperate to provide helical paths around and along the passageway or aperture of the plate.

In the illustrated embodiment, the implant assembly 110 includes two screws 114 for rotatably driving into a respective aperture 124 of the plate 112 and into the bone of the patient. Each of the screws 114 includes a head 134 and a threaded body 136 extending from the head 134. The body 136 has a body thread or threads 142 formed therealong. The body thread 142 is sized to be received through the central aperture 126 of the plate and is configured to thread into the bone of the patient. The head 134 includes a drive or socket 140 that is configured to receive or otherwise engage a tool or driver to rotatably drive the screw 114 into the bone and plate.

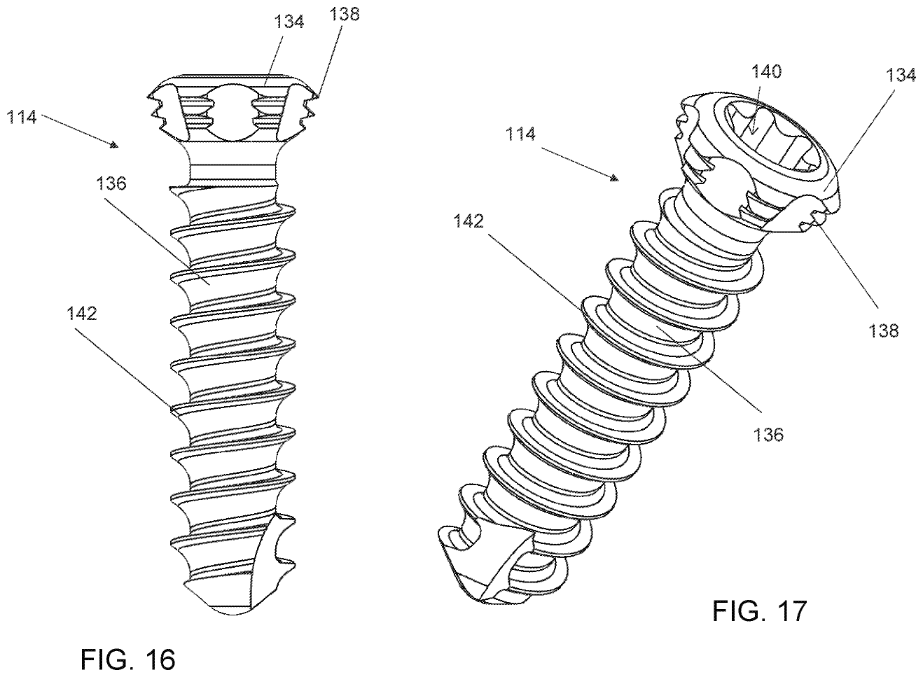

The head 134 also includes a plurality of spaced apart protrusions or buttresses 138 protruding radially outward (such as six sets of protrusions spaced around the head of the screw). The buttresses or steps or parapets or castellations 138 are vertically spaced apart between the top and bottom of the head portion of the screw. The buttresses 138 are parallel to one another and are generally perpendicular to the longitudinal axis of the screw. In other words, and as can be seen with reference to FIG. 16, each buttress 138 comprises a tapered tooth or ridge that has an upper surface and a lower surface that meet at an edge, with the edges of the buttresses being parallel to one another and perpendicular to the longitudinal axis of the screw. That is, the edges of the buttresses are not angled relative to the longitudinal axis of the screw and thus do not extend in a swirling or helical configuration found in typical threads. The buttresses are vertically spaced apart from one another and are circumferentially spaced from other buttresses or sets of buttresses and allow the screw to rotatably engage the partial threads of the plate.

The head buttresses 138 are configured to engage the partial threads 130 of the plate when the screw 114 is inserted and rotatably driven through the aperture 124 of the plate 112. The spaced apart buttresses of the screw head and the spaced apart partial threads of the plate engage one another in a manner that allows the screw to be rotatably driven through the plate in a direction perpendicular to the plate or at an acute angle relative to the plate, such as in a manner similar as described above. The screws 114 and plate 112 may be otherwise similar to the screws 14 and plate 12, discussed above, such that a detailed description of the screws and plates need not be repeated herein.

The foregoing description has been provided for purposes of illustration and description. It is not intended to be exhaustive or to limit the disclosure. Individual elements or features of a particular configuration are generally not limited to that particular configuration, but, where applicable, are interchangeable and can be used in a selected configuration, even if not specifically shown or described. The same may also be varied in many ways. Such variations are not to be regarded as a departure from the disclosure, and all such modifications are intended to be included within the scope of the disclosure.

1. A surgical implant assembly comprising:
a plate comprising at least one aperture extending through the plate between opposite surfaces of the plate and having a central axis extending along the aperture between the opposite surfaces of the plate;
a screw comprising a head and a body, the head including at least one head thread configured to engage with the plate at the aperture and the body including at least one body thread configured to engage a bone material;
wherein the at least one head thread has a first pitch and the at least one body thread has a second pitch that is different than the first pitch;
wherein the first pitch of the at least one head thread is greater than the second pitch of the at least one body thread; and
wherein, when the plate is disposed at a surface of the bone material, the pitch difference between the first pitch of the at least one head thread of the head and the second pitch of the at least one body thread of the body causes the plate to lift away from the surface of the bone material as the screw is rotatably driven into the bone material.

2. The surgical implant assembly of claim 1, wherein the plate comprises a plurality of spaced apart buttresses established at and extending radially into the at least one aperture, wherein the plurality of buttresses are arranged as a plurality of circumferentially spaced apart sets of buttresses, and wherein each set of buttresses comprises at least two axially spaced apart buttresses that are spaced apart between the opposite surfaces of the plate, and wherein the at least one head thread engages the buttresses of the plate as the screw is rotatably driven into the bone material.

3. The surgical implant assembly of claim 1, wherein the at least one head thread comprises a plurality of head threads.

4. The surgical implant assembly of claim 3, wherein the screw has a longitudinal axis, and wherein, when the head threads are engaged with the plate and the screw is driven into the bone material, the longitudinal axis of the screw is not parallel to the central axis of the at least one aperture.

5. The surgical implant assembly of claim 1, wherein the first pitch of the at least one head thread is greater than 1.5 mm and the second pitch of the body thread is less than 1.5 mm.

6. The surgical implant assembly of claim 1, wherein the first pitch of the at least one head thread is between 1.5 mm and 2.5 mm and the second pitch of the at least one body thread is between 0.9 mm and 1.5 mm.

7. The surgical implant assembly of claim 1, comprising a second screw having a head and a body, the head including at least one head thread configured to engage with the plate at the aperture and the body including at least one body thread configured to engage the bone material, wherein the at least one head thread of the second screw has a first pitch and the at least one body thread of the second screw has a second pitch that is different than the first pitch of the at least one head thread of the second screw, wherein the first pitch of the at least one head thread of the second screw is less than the second pitch of the at least one body thread of the second screw, and wherein, when the plate is disposed at the surface of the bone material, the pitch difference between the first pitch of the at least one head thread of the head of the second screw and the second pitch of the at least one body thread of the body of the second screw causes the plate to compress down onto the surface of the bone material as the second screw is rotatably driven into the bone material.

8. The surgical implant assembly of claim 1, comprising a second screw having a head and a body, the head including at least one head thread configured to engage with the plate at the aperture and the body including at least one body thread configured to engage the bone material, wherein the at least one head thread of the second screw has a first pitch and the at least one body thread of the second screw has a second pitch that is the same as the first pitch of the at least one head thread of the second screw, and wherein, when the plate is disposed at the surface of the bone material, the first pitch of the at least one head thread of the head of the second screw and the second pitch of the at least one body thread of the body of the second screw enables the plate to neither lift off of or compress onto the surface of the bone material as the second screw is rotatably driven into the bone material.

9. A surgical implant system comprising:
a plate comprising at least one aperture extending through the plate between opposite surfaces of the plate and having a central axis extending along the aperture between the opposite surfaces of the plate;
a first screw comprising a first head and a first body, the first head including at least one first head thread configured to engage with the plate at the aperture and the first body including at least one first body thread configured to engage a bone material;
wherein the at least one first head thread has a first head pitch and the at least one first body thread has a first body pitch that is different than the first head pitch, and wherein the first head pitch is greater than the first body pitch;
a second screw comprising a second head and a second body, the second head including at least one second head thread configured to engage with the plate at the aperture and the second body including at least one second body thread configured to engage the bone material;
wherein the at least one second head thread has a second head pitch and the at least one second body thread has a second body pitch that is different than the second head pitch, and wherein the second body pitch is greater than the second head pitch;
wherein, when the plate is disposed at a surface of the bone material and the first screw is selected and disposed at the aperture, the pitch difference between the first head pitch and the first body pitch causes the plate to lift away from the surface of the bone material as the first screw is rotatably driven into the bone material; and
wherein, when the plate is disposed at the surface of the bone material and the second screw is selected and disposed at the aperture, the pitch difference between the second head pitch and the second body pitch causes the plate to compress onto the surface of the bone material as the second screw is rotatably driven into the bone material.

10. The surgical implant system of claim 9, further comprising a third screw comprising a third head and a third body, the third head including at least one third head thread configured to engage with the plate at the aperture and the third body including at least one third body thread configured to engage the bone material, and wherein the at least one third head thread has a third head pitch and the at least one third body thread has a third body pitch that is the same as the third head pitch, and wherein, when the plate is disposed at the surface of the bone material and the third screw is selected and disposed at the aperture, the third head pitch and the third body pitch enables the plate to neither compress down onto the surface of the bone material nor lift away from the surface of the bone material as the third screw is rotatably driven into the bone material.

11. The surgical implant system of claim 9, wherein the at least one first head thread comprises a plurality of first head threads, and wherein the plurality of first head threads provides a plurality of thread entry points for threading into the aperture of the plate.

12. The surgical implant system of claim 11, wherein the plurality of first head threads, when the plate is disposed at the surface of the bone material and the first screw is rotatably driven into the bone material, enable the first screw to engage the plate with a longitudinal axis of the first screw being at an angle relative to the central axis of the aperture.

13. The surgical implant system of claim 11, wherein the plate comprises a plurality of spaced apart buttresses established at and extending radially into the at least one aperture, wherein the plurality of buttresses are arranged as a plurality of circumferentially spaced apart sets of buttresses, and wherein each set of buttresses comprises at least two axially spaced apart buttresses that are spaced apart between the opposite surfaces of the plate, and wherein the first head threads engage the buttresses of the plate when the first screw is selected and disposed at the aperture and rotatably driven into the bone material.

14. The surgical implant system of claim 9, wherein the plate comprises a plurality of spaced apart buttresses established at and extending radially into the at least one aperture, wherein the plurality of buttresses are arranged as a plurality of circumferentially spaced apart sets of buttresses, and wherein each set of buttresses comprises at least two axially spaced apart buttresses that are spaced apart between the opposite surfaces of the plate, and wherein the at least one first head thread engages the buttresses of the plate when the first screw is selected and disposed at the aperture and rotatably driven into the bone material.

15. A method for mending bone using a surgical implant assembly, the method comprising:
providing a plate comprising at least one aperture extending through the plate between opposite surfaces of the plate and having a central axis extending along the aperture between the opposite surfaces of the plate;
providing a first screw comprising a first head and a first body, the first head including at least one first head thread configured to engage with the plate at the aperture and the first body including at least one first body thread configured to engage a bone material, wherein the at least one first head thread has a first head pitch and the at least one first body thread has a first body pitch that is different than the first head pitch, and wherein the first head pitch is greater than the first body pitch;
providing a second screw comprising a second head and a second body, the second head including at least one second head thread configured to engage with the plate at the aperture and the second body including at least one second body thread configured to engage the bone material, wherein the at least one second head thread has a second head pitch and the at least one second body thread has a second body pitch that is different than the second head pitch, and wherein the second body pitch is greater than the second head pitch;
disposing the plate at a surface of the bone material;
selecting one of the first screw or the second screw;
disposing the selected one of the first screw or the second screw at the aperture;
rotatably driving the selected one of the first screw or the second screw into the bone material;
wherein, responsive to the first screw being selected and disposed at the aperture, the pitch difference between the first head pitch and the first body pitch causes the plate to lift away from the surface of the bone material as the selected first screw is rotatably driven into the bone material; and
wherein, responsive to the second screw being selected and disposed at the aperture, the pitch difference between the second head pitch and the second body pitch causes the plate to compress onto the surface of the bone material as the selected second screw is rotatably driven into the bone material.

16. The method of claim 15, further comprising providing a third screw comprising a third head and a third body, the third head including at least one third head thread configured to engage with the plate at the aperture and the third body including at least one third body thread configured to engage the bone material, wherein the at least one third head thread has a third head pitch and the at least one third body thread has a third body pitch that is the same as the third head pitch, and wherein, responsive to the third screw being selected and disposed at the aperture, the plate neither compresses down onto the surface of the bone material nor lifts away from the surface of the bone material as the third screw is rotatably driven into the bone material.

17. The method of claim 15, wherein the at least one first head thread comprises a plurality of first head threads, and wherein the plurality of first head threads provides a plurality of thread entry points for threading into the aperture of the plate.

18. The method of claim 17, wherein the plurality of first head threads, when the plate is disposed at the surface of the bone material and the first screw is rotatably driven into the bone material, enable the first screw to engage the plate with a longitudinal axis of the first screw being at an angle relative to the central axis of the aperture.

19. The method of claim 17, wherein the plate comprises a plurality of spaced apart buttresses established at and extending radially into the at least one aperture, wherein the plurality of buttresses are arranged as a plurality of circumferentially spaced apart sets of buttresses, and wherein each set of buttresses comprises at least two axially spaced apart buttresses that are spaced apart between the opposite surfaces of the plate, and wherein the first head threads engage the buttresses of the plate when the first screw is selected and disposed at the aperture and rotatably driven into the bone material.

20. The method of claim 15, wherein the plate comprises a plurality of spaced apart buttresses established at and extending radially into the at least one aperture, wherein the plurality of buttresses are arranged as a plurality of circumferentially spaced apart sets of buttresses, and wherein each set of buttresses comprises at least two axially spaced apart buttresses that are spaced apart between the opposite surfaces of the plate, and wherein the at least one first head thread engages the buttresses of the plate when the first screw is selected and disposed at the aperture and rotatably driven into the bone material.
